(19) United States
(12) Patent Application Publication
(10) Pub. No.: US 2026/0248461 A1
(43) Pub. Date: Aug. 27, 2026

(54) METHOD OF PREPROCESSING HEIGHT MEASUREMENT DATA FOR GROWTH PREDICTION

(71) Applicant: GP CO., LTD., Gwangmyeong-si, Gyeonggi-do (KR)

(72) Inventors: Je Hyeok Seong, Gwangmyeong-si, Gyeonggi-do (KR); Ji Hun Kim, Gwacheon-si, Gyeonggi-do (KR); Do Hyun Chun, Seoul (KR); Jong Ho Kang, Seoul (KR)

(73) Assignee: GP CO., LTD., Gwangmyeong-si, Gyeonggi-do (KR)

(21) Appl. No.: 18/863,880

(22) PCT Filed: Jun. 14, 2024
(86) PCT No.: PCT/KR2024/008204

§ 371 Date: Nov. 7, 2024

(30) Foreign Application Priority Data
Aug. 30, 2023 (KR) 10-2023-0114699

(51) Int. Cl. A61B 5/00; A61B 5/107

(52) U.S. Cl. CPC A61B 5/7275; A61B 5/1072; A61B 5/7264; A61B 2503/06

(57) ABSTRACT

The present disclosure relates to a method, apparatus, and computer program for preprocessing input data for predicting growth of children or adolescents. A method of preprocessing height measurement data for growth prediction according to an exemplary embodiment of the present disclosure may include receiving time-series bio-component data of a subject; receiving identification data of the subject, extracting height data from the bio-component data, determining whether there is a decreasing section within a section where the height data is input, and when there is the section where the height data decreases, determining an error in the height data based on a growth stage in which the decreasing section is included.

### TECHNICAL FIELD The present disclosure relates to a method, apparatus, and computer program for preprocessing input data for predicting growth of children or adolescents.

### BACKGROUND ART

With the recent development of artificial intelligence technologies, the artificial intelligence technologies are being applied to various fields. Instead of existing data processing methods, methods of generating additional information by extracting features inherent in data through neural network models have been developed and used.

Currently, the artificial intelligence technology has gone beyond simply tracking and detecting objects and is also being applied to train a past history and derive current features that reflect future predictions or time-series change information.

In addition, in artificial intelligence that trains data, the quality of data is known to be one of the key factors in the performance of artificial intelligence. For example, leading companies with the most advanced autonomous driving technology based on the artificial intelligence are emphasizing that securing high-quality data is key.

Among these, the predictive analysis is a technology in areas Of statistics and data mining that extracts information from data and uses the extracted information to predict trends and behavior patterns. This predictive analysis may be applied to all areas where decisions are needed based on information obtained from data. The core of predictive analysis is understanding the relationships between variables and then predicting unknown variables.

For this purpose, various approaches are being used depending on the data characteristics and prediction target.

Among various fields that require the predictive analysis, there is the field of physical growth in children and adolescents. There is a lot of interest among parents and adolescents about when height growth will occur and how much growth will occur.

Regarding the conventional prediction of height growth, a method of predicting a growth plate by taking an X-ray or analyzing a relationship with genetic/environmental factors has been proposed (Korean Patent No. 10-2075743, Korean Patent No. 10-1866208), and a method of making physical data of sample subjects having different measurement times or number of measurements into a form suitable for training a growth prediction model has been proposed (Korean Patent No. 10-2198302).

Since the children and adolescents have growth stages with different features, it is possible to increase the reliability of providing solutions through predicted data and analysis by considering the growth states.

In particular, since the children and adolescents include a period of rapid physical change according to the growth stage, it is necessary to first determine the errors in the input (or measurement) data (bio-component data) that can secure the high-quality data to increase the accuracy of growth prediction and the performance of the artificial intelligence learning.

### DISCLOSURE

### Technical Problem Accordingly, an object of the present disclosure is to solve the above problems.

One of the various tasks of the present disclosure provides a method, apparatus, and computer program for preprocessing input data for predicting growth of children and adolescents, providing customized solutions for each growth stage, etc.

### Technical Solution

According to an aspect of the present disclosure, a method of preprocessing height measurement data for growth prediction performed by computing device includes: receiving time-series bio-component data of a subject; receiving identification data of the subject; extracting height data from the bio-component data; determining whether there is a decreasing section within a section where the height data is input; and when there is the section where the height data decreases, determining an error in the height data based on a growth stage in which the decreasing section is included.

The method may further include generating first data by concatenating the time-series bio-component data of the subject and the identification data of the subject.

The method may further include after generating the first data, deleting the bio-component data when a preset value is included in the bio-component data.

The growth stage may include a normal growth period, a rapid growth period, a decelerated growth period, and a non-growth period, and may be classified based on a monthly age of the identification data.

In the determining of the error, when the decreasing section is included in the rapid growth period, the height data of the decreasing section may be deleted.

The determining of the error may include calculating a difference between height data at any first point and height data at a second point, respectively, forming the decreasing section and an average data value of the corresponding monthly age of a plurality of sample subjects, when the decreasing section is included in the normal growth period.

The determining of the error may further include comparing the difference value calculated at the first point with the difference value calculated at the second point.

In the determining of the error, when the difference value calculated at the first point is smaller than the difference value calculated at the second point, the height data at the second point may be deleted.

In the determining of the error, when the difference value calculated at the first point is greater than or equal to the difference value calculated at the second point, the height data at the first point may be deleted.

In the determining of the error, when the decreasing section is included in the decelerated growth period or the non-growth period, the data of the decreasing section may be deleted based on a period of the decreasing section and a degree of decrease in height.

A program stored in a computer-readable recording medium including a program code for executing the method of preprocessing height measurement data described above may be provided.

A computer-readable recording medium on which a program for executing the method of preprocessing height measurement data for growth prediction described above may be provided.

According to another aspect of the present disclosure, an apparatus for preprocessing height measurement data for growth prediction includes: a first input unit that receives time-series bio-component data of a subject; a second input unit that receives identification data of the subject; a height data extraction unit that extracts height data from the bio-component data; a decreasing section determination unit that determines whether there is a decreasing section within a section where the height data is input; a growth stage classification unit that classifies a growth stage including the decreasing section when there is the section where the height data decreases; and an error determination unit that determines an error in the height data based on the growth stage including the decreasing section.

The apparatus may further include a connection unit that connects data input through the first input unit and the second input unit to generate first data.

The apparatus may further include an error detection unit that deletes the bio-component data when a preset value is measured in the bio-component data among the first data.

Each feature of the above-described embodiments may be implemented in combination in other embodiments unless inconsistent with or exclusive of the other embodiments.

### Advantageous Effects

According to various embodiments of the present disclosure, when predicting the growth of children and adolescents, by determining the errors in bio-component data measured for predicting the growth of children and adolescents, it is possible to increase the accuracy of growth prediction.

According to various embodiments of the present disclosure, by removing error data that may occur when measuring a height of a subject by determining a section where a height decreases, it is possible to increase the accuracy of growth prediction. In addition, when determining errors in the section where the height decreases, by considering the growth stage of children and adolescents, it is possible to more efficiently determine error data.

According to various embodiments of the present disclosure, when providing a solution necessary for the growth of children and adolescents, by eliminating errors in input data, it is possible to increase the accuracy of growth prediction.

According to various embodiments of the present disclosure, by determining errors in measured bio-component data for predicting the growth of children and adolescents to generate refined input data, it is possible to increase the accuracy of growth prediction of children and adolescents.

According to various embodiments of the present disclosure, by determining errors in measured bio-component data for predicting the growth of children and adolescents to generate refined input data, it is possible to increase the accuracy of solutions necessary for children and adolescents.

The effects of the present disclosure are not limited to the above-mentioned effects, and other effects that are not mentioned may be obviously understood by those skilled in the art from the following description.

### BRIEF DESCRIPTION OF THE DRAWINGS

FIG. 1 is a diagram illustrating the configuration of a growth prediction and solution generation device according to an exemplary embodiment of the present disclosure.

FIG. 2 is a diagram illustrating a data preprocessing unit of FIG. 1.

FIG. 3 is a diagram illustrating a predicted height and target height for each growth stage according to an exemplary embodiment of the present disclosure.

FIG. 4 is a diagram illustrating the growth in obesity and normal weight for each growth stage of a boy according to an exemplary embodiment of the present disclosure.

FIG. 5 is a diagram illustrating the growth in obesity and normal weight for each growth stage of a girl according to an exemplary embodiment of the present disclosure.

FIG. 6 is a diagram illustrating a configuration of a neural network for performing growth prediction and solution provision according to an exemplary embodiment of the present disclosure.

FIG. 7 is a diagram illustrating a first neural network model according to an exemplary embodiment of the present disclosure.

FIG. 8 is a diagram illustrating a second neural network model according to an exemplary embodiment of the present disclosure.

FIGS. 9 to 11 are diagrams illustrating a method of preprocessing height measurement data for growth prediction according to an exemplary embodiment of the present disclosure.

FIGS. 12 and 13 are diagrams visually illustrating a process of preprocessing input data according to an exemplary embodiment of the present disclosure.

FIG. 14 is a diagram illustrating bio-component data of a subject according to an exemplary embodiment of the present disclosure.

FIG. 15 is a diagram illustrating data concatenating bio-component data and identification data of a subject according to an exemplary embodiment of the present disclosure.

### BEST MODE

Hereinafter, detailed embodiments of the present disclosure will be described with reference to the accompanying drawings. The following detailed descriptions are provided to help a comprehensive understanding of methods, devices and/or systems described herein. However, the embodiments are described by way of examples only and the present disclosure is not limited thereto.

In describing exemplary embodiments of the present disclosure, when it is decided that a detailed description of a well-known technology related to the present disclosure may unnecessarily obscure the gist of the present disclosure, the detailed description will be omitted. Further, the following terminologies are defined in consideration of the functions in the present disclosure and may be construed in different ways by the intention of users and operators. Therefore, the definitions thereof should be construed based on the contents throughout the specification.

The terms used in the detailed description is merely for describing the embodiments of the present disclosure and should in no way be limited. Unless explicitly used otherwise, expressions in a singular form include the meaning in a plural form.

In the present description, expressions such as “include” or “comprise” are used to refer to certain features, numbers, steps, operations, components, or some or a combination thereof, and should not be construed to preclude the presence or addition of one or more other features, numerals, steps, operations, components other than those described, or some or a combination thereof.

In addition, terms ‘first’, ‘second’, A, B, (a), (b), and the like, will be used in describing components of exemplary embodiments of the present disclosure. These terms are used only to differentiate the components from other components. Therefore, the nature, times, sequence, etc. of the corresponding components are not limited by these terms.

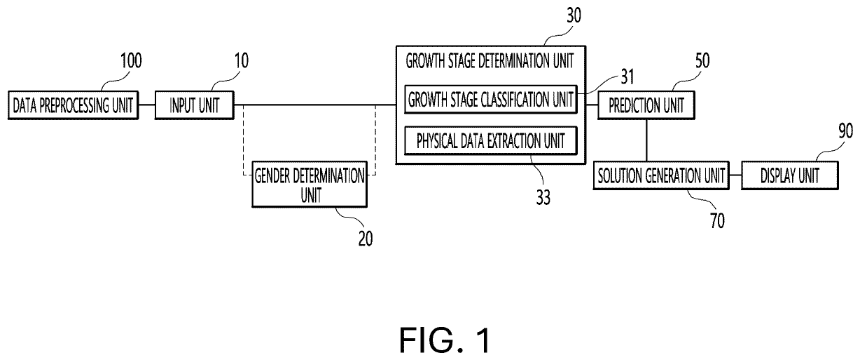

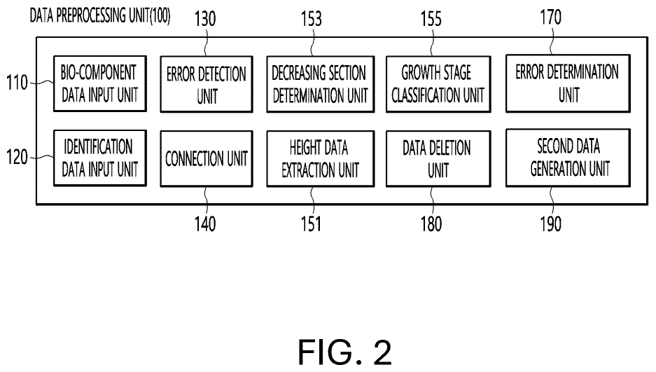

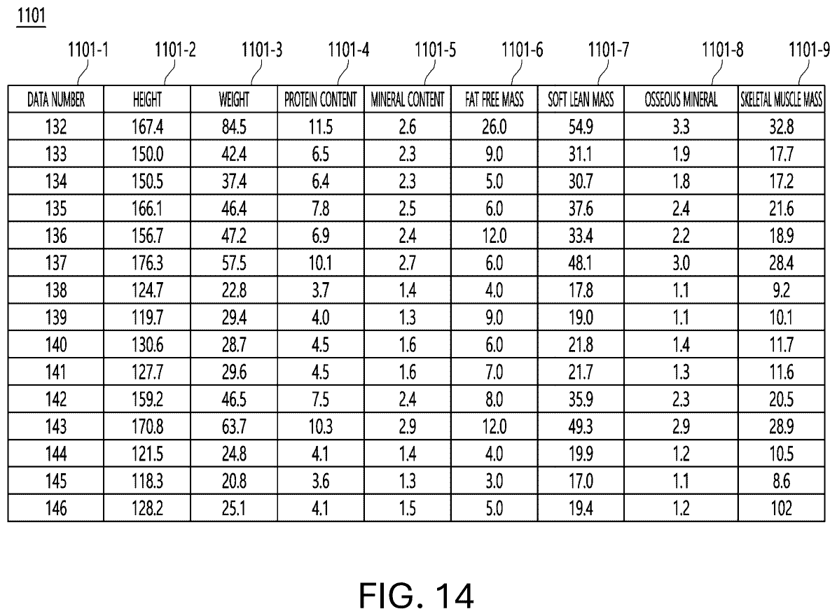

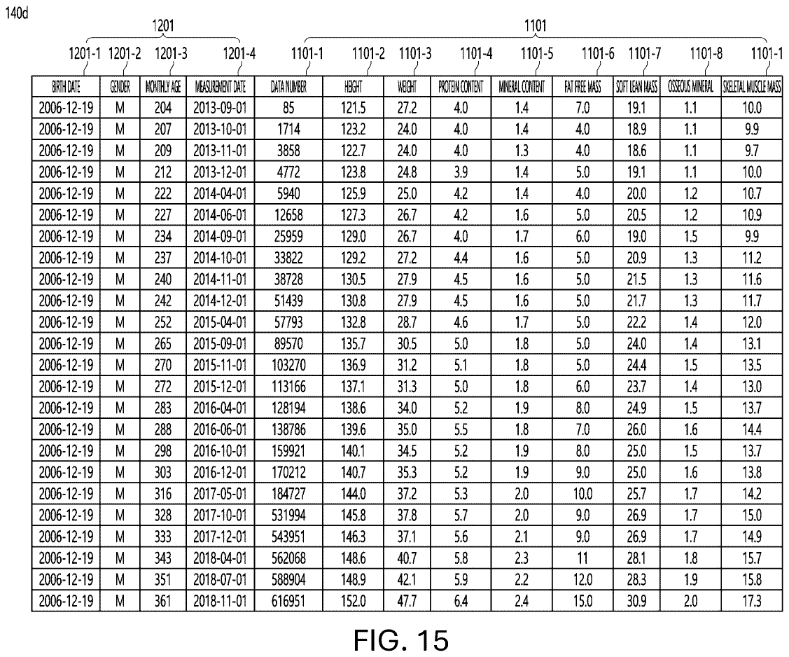

FIG. 1 is a diagram illustrating a configuration of a growth prediction and solution generation device according to an exemplary embodiment of the present disclosure, FIG. 2 is a diagram illustrating a data preprocessing unit of FIG. 1, FIG. 14 is a diagram illustrating bio-component data of a subject according to an exemplary embodiment of the present disclosure, and FIG. 15 is a diagram illustrating data concatenating bio-component data and identification data of a subject according to an exemplary embodiment of the present disclosure.

Hereinafter, the description will be made with reference to FIGS. 1, 2, 14, and 15.

A growth prediction system of the present embodiment may preprocess and then receive time-series physical data of a subject. A gender of a subject may be distinguished based on input physical data, and growth prediction or a solution through growth prediction may be generated through a different growth prediction model depending on the gender of the subject.

In the case of boys and girls, the growth rate by growth stage may be different. For example, as described below, boys and girls may each enter a rapid growth stage at different times, and thus the growth rate, the generation of solutions considering the growth rate, etc., may be different. Therefore, it is desirable to perform growth prediction using a model trained through different learning data based on the gender of the subject.

More specifically, the growth prediction and solution generation device according to an exemplary embodiment of the present disclosure may include a data preprocessing unit 100, an input unit 10, a gender determination unit 20, a growth stage determination unit 30, a prediction unit 50, a solution generation unit 70, and a display unit 90.

Through the input unit 10, the growth prediction and solution generation device may receive the time-series physical data of the subject.

The physical data of the subject may include data (identification data) that may identify the subject and the bio-component data of the subject. The identification data and the bio-component data may be composed of the time-series data.

As an example, the identification data may include data for identifying a subject such as grade (or age), gender, and height, and the bio-component data may include data such as weight, protein, mineral content, body fat, body water, soft lean mass, fat free mass, bone tissue, skeletal muscle mass, body mass index (BMI), basal metabolic rate, neck circumference, chest circumference, abdominal circumference, thigh circumference, arm circumference, and hip circumference.

More specifically, referring to FIGS. 14 and 15, identification data 1201 of a subject may include a birth date 1201-1, a gender 1201-2, a monthly age 1201-3, and a measurement date 1201-4.

In addition, a bio-component data 1101 of the subject may include a data number 1101-1, a height 1101-2, weight 1101-3, a protein content 1101-4, a mineral content 1101-5, a fat free mass 1101-6 (body fat mass), a soft lean mass 1101-7, osseous mineral 1101-8, and a skeletal muscle mass 1101-9.

The physical data is only an example to help understand the present disclosure, and the present embodiment is not limited thereto. Of course, the types of information constituting the physical data may be changed in various ways according to the embodiment.

Meanwhile, an exemplary embodiment of the present disclosure may include the data preprocessing unit 100 that determines an error in the body data of the subject to generate refined data.

Since bio-component data 1101 may undergo rapid changes depending on the growth stage due to the physical characteristics of children and adolescents, errors (errors in the measurement process) may occur in other bio-component data measurement processes such as a measurement method, a measurement time, and a measurement environment.

In addition, errors (data transmission errors) may occur in the process of transmitting or storing the measured bio-component data. Therefore, after the data preprocessing unit 100 of the present embodiment determines whether there is a section where the height 1101-2 decreases in the measured bio-component data 1101, when there is a section where the height data decreases, the data processing unit 100 may determine an error in the height data 1101-2 by considering the growth stage of the subject through the monthly age data 1201-3 in the identification data 1201.

The refined data may be generated through a process of determining an error in the measurement process and an error in data transmission when the section where the height data 1101-2 decreases among the bio-component data 1101 as described above.

The data preprocessing unit 100 of the present embodiment may determine the error in the input bio-component data 1101 and identification data 1201 as described above to generate the refined data (second data). The second data generated in this way may be used as input data of the input unit 10 for predicting the growth of the subject or generating a solution through growth prediction.

Meanwhile, the data preprocessing unit 100 can include a bio-component data input unit 110, an identification data input unit 120, an error detection unit 130, a connection unit 140, a height data extraction unit 151, a decreasing section determination unit 153, a growth stage classification unit 155, a data deletion unit 180, and a second data generation unit 190.

The data preprocessing unit 100 may receive the bio-component data 1101 of the subject through the bio-component data input unit 110, and receive the identification data 1201 of the subject through the identification data input unit 120.

The error detection unit 130 may detect whether there is a preset value among the input bio-component data 1101 of the subject, and the present value may be set to a ‘Nan’ value, which is an example of a measurement error.

The connection unit 140 may generate first data 140d by concatenating the bio-component data 1101 of the subject and the identification data 1201. The first data 140d is data before being refined through an error determination unit 170, and may be distinguished from second data refined through the error determination unit 170.

The height data extraction unit 151 may extract the height data 1101-2 from the input bio-component data 1101 of the subject, and determine whether the decreasing section exists in the height data extracted through the decreasing section determination unit 153.

The growth stage classification unit 155 may classify the growth stage of the subject based on the identification data 1201 of the input subject.

The error determination unit 170 may determine the error in the height data when it is determined that the decreasing section exists in the height data extracted through the decreasing section determination unit 153.

More specifically, the criterion for determining the error in the height data may be determined differently based on the growth stage in which the section where the height data decreases is included, based on the growth stage of the subject classified through the growth stage classification unit 155 described above.

The data deletion unit 180 may delete data determined to be an error through the error determination unit 170, and the second data generation unit 190 may determine the error in the input data and then generate the refined data (second data).

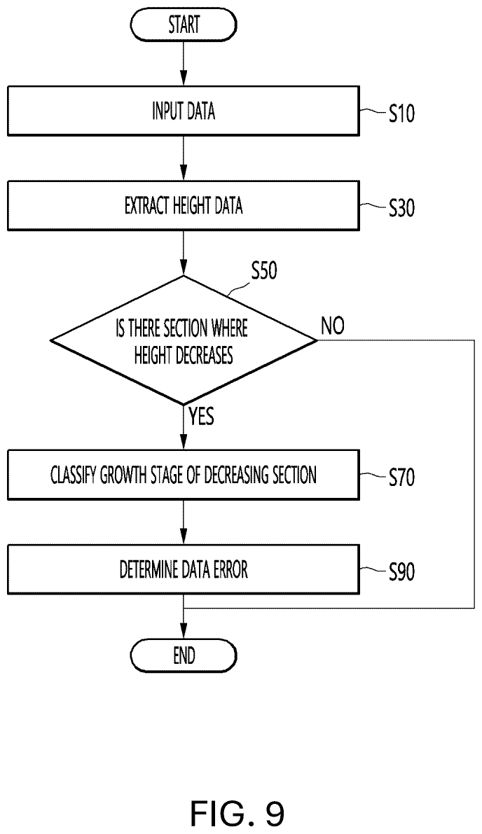

The method of preprocessing height measurement data for growth prediction through the above-described data preprocessing unit will be described in more detail with reference to FIGS. 9 to 11.

Meanwhile, the data input to the input unit 10 may include the time-series physical data of the subject.

The time-series physical data may be continuous data or discontinuous data, but may be data included in at least one period corresponding to the growth stage of children and adolescents.

More specifically, the collection period and the number of times of collections of the time-series physical data of the subject may vary.

For example, a first subject may have measured physical data from ages 8 to 12, which is part of the children and adolescents period, and a second subject may have irregularly measured physical data such as ages 8, 10 to 12, and 15.

In addition, a third subject may have physical data measured multiple times during a certain period (period corresponding to one of the growth stages of children and adolescents), while a fourth subject may have physical data measured only once during a certain period (period corresponding to one of the growth stages of children and adolescents).

As described above, depending on the collection period and the number of times of collections, the physical data of the subject may be included in two or more of the growth stages of children and adolescents (the first subject, the second subject), but may not be included therein (the third subject and the fourth subject).

As in the third subject, when there is the physical data measured multiple times during one of the plurality of growth stages, the growth stage determination unit 30 may classify the growth stage in which the physical data of the third subject is included through a growth stage classification unit 31, and extract the physical data through a physical data extraction unit 33. Here, the extracted physical data may generally include all of the physical data of the third subject input through the input unit 10.

However, as in the fourth subject, when the physical data of the subject corresponds to only one of the plurality of growth stages, and there is physical data measured only once during that period, physical data corresponding to an arbitrary period may further be generated before the physical data of the fourth subject is input to the growth stage determination unit 30.

More specifically, time-series physical data of a subject corresponding to an arbitrary period may be generated based on the time-series physical growth data of the plurality of sample subjects in which the input physical data of the subject have been previously stored.

As an example, the physical data may be generated based on a distribution model (similarity) between the input physical data of the subject and the pre-stored time series physical growth data of the plurality of sample subjects, or the physical data may be generated based on a Bayesian inference model (conditional probability).

The growth stage determination unit 30 may classify the growth stage into one of the plurality of growth stages in the growth stage classification unit 31 based on the physical data of the subject input through the input unit 10 and extract physical data corresponding to the classified growth stage in the physical data extraction unit 33.

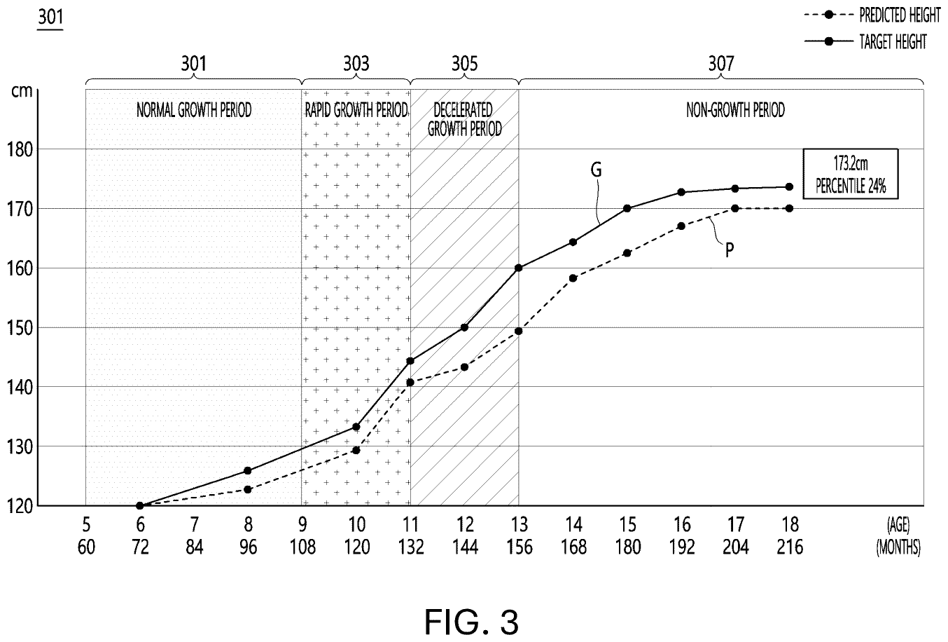

FIG. 3 is a diagram illustrating a predicted height and target height by growth stage according to an exemplary embodiment of the present disclosure.

In FIG. 3, an x-axis represents age and months, and a y-axis represents height (cm). A dotted line P below represents a predicted growth rate of the subject, and a solid line G above represents a target growth rate of an evaluation subject.

Referring to FIG. 3 and describing the growth stage of children and adolescents described above, the growth stage may include a normal growth period 301, a rapid growth period 303, a decelerated growth period 305, and a non-growth period 307.

Each growth stage may be classified according to the growth rate, and the height grown each year varies depending on each growth stage, and the actual height grown even in the same growth stage may vary depending on the growth type.

The normal growth period 301 generally refers to the period before puberty when secondary sexual characteristics appear. The children and adolescents corresponding to this period generally have open growth plates. As a result, depending on the growth environment, in the case of a short height growth type, a height generally grows by 4 to 5 cm per year, and in the case of a tall growth type, a height grows in the range of 6 to 7 cm per year.

The rapid growth period 303 is a period in which secondary sexual characteristics begin to appear. In women, a breast swells and a lump appears, and in men, the testicles grow larger, pubic hair begins to grow, and a voice break appears where voice changes. The rapid growth period 303 generally lasts about 2 to 3 years after the normal growth period 301, and a height grows in the range of 7 to 10 cm per year on average.

The decelerated growth period 305 refers to a period in which the secondary sexual characteristics are completed. During this period, in the case of women, the secondary sexual characteristics may be clearly identified starting from menarche, and in the case of men, the secondary sexual characteristics may be clearly identified through pubic hair, voice change, and armpit hair. In the decelerated growth period 305, the growth rate drops rapidly compared to the rapid growth period 303. The decelerated growth period 305 generally lasts about 2 to 3 years, and a height grows in the range of 5 to 6 cm per year on average, and does not naturally grow any further.

The growth plate begins to close little by little after the rapid growth period 304, and closes approximately 50% about 6 months after entering the decelerated growth period 305.

The non-growth period 307 refers to a period in which the growth plate has closed, as a period in which the growth period has not completely ended but natural height growth has become difficult. Generally, women enter the non-growth period 307 about 1 year and 6 months to 2 years after menarche, and men enter the non-growth period 307 about 1 year and 6 months to 2 years from the time hair begins to appear in armpits. In the non-growth period 307, the growth plate closes and the natural growth stops, but by changing bad lifestyle habits and improving a physical function through customized exercise, posture correction, and nutrient intake, etc., a height may grow in the range of about 1 to 3 cm.

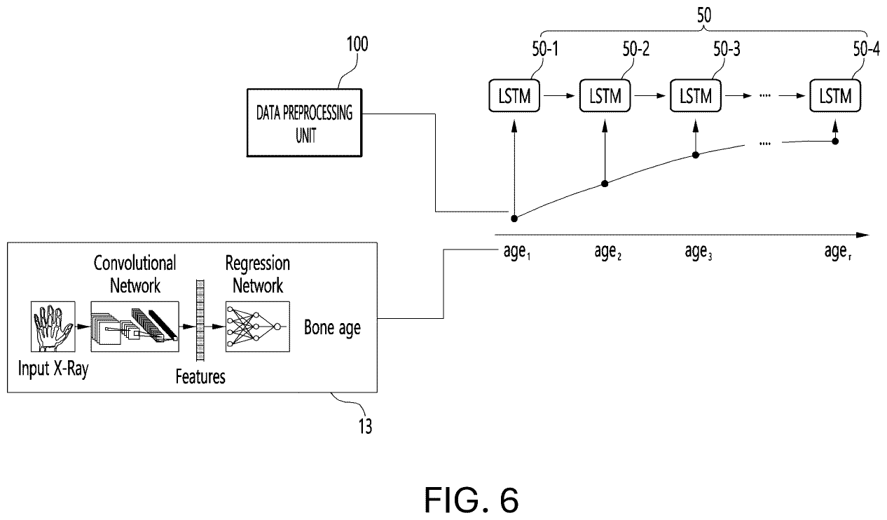

Meanwhile, the prediction unit 50 is a prediction model and may be implemented with artificial intelligence in a recursive neural network (RNN) structure so that it may use not only current values but also time series values. For example, the prediction model may be implemented with architecture such as Long Short Term Memory (LSTM), or Gated Recurrent Units (GRU) that is the RNN. Of course, in addition to this, conventional various artificial intelligence architectures may be applied to the prediction model of this embodiment, which will be described in detail with reference to FIGS. 6 to 8 described later.

The solution generation unit 70 may generate a growth management solution based on the physical data of the subject corresponding to the classified growth stage.

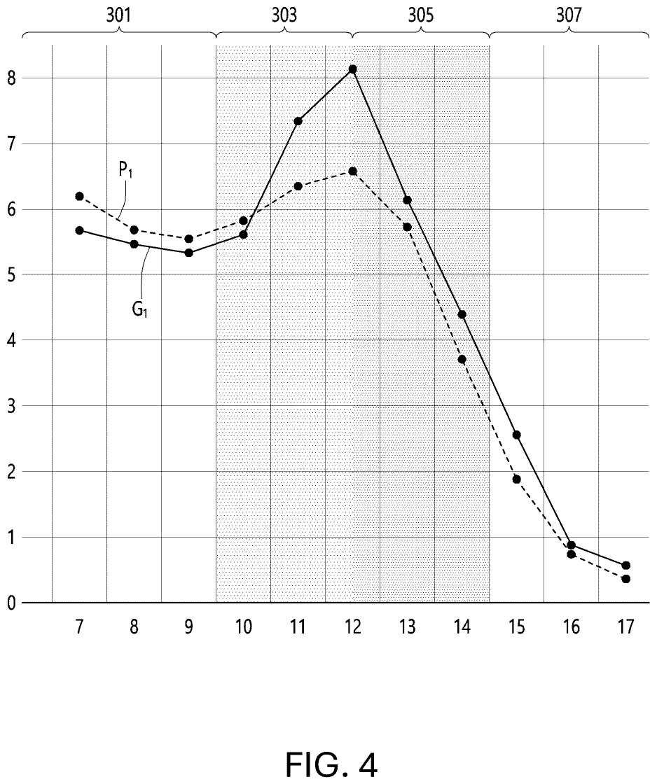

More specifically, when the subject corresponds to the normal growth period 301, a solution for increasing the growth prediction value of the subject may be provided. The growth prediction value is a value corresponding to the y-axis in FIG. 4, and the solution for increasing the growth prediction value may be provided to the subject through various solution display units 90 for increasing the expected target value of the y-axis.

Meanwhile, examples of solutions provided through the display unit 90 may include a current height, a predicted height, an obesity level, a fat free mass, a skeletal muscle mass, a protein content, a mineral content, an amount of sleep, an amount of exercise, nutritional information, lifestyle habits, posture, etc. Each indicator may be expressed step by step as caution, normal, good, etc., based on a preset range, or may also be expressed as a level.

The current state, customized solutions, precautions, etc., for each indicator may be displayed. The current state may be displayed step by step or level based on the target value. The customized solutions may include contents for adjustment of protein, mineral content, body fat, body water, soft lean mass, fat free mass, bone tissue, skeletal muscle mass, body mass index (BMI), basal metabolic rate, etc., to reach the current target value based on the input physical data.

The precautions may include contents for adjustment of the current insufficient amount of protein, mineral content, body fat, body water, soft lean mass, fat free mass, bone tissue, skeletal muscle mass, body mass index (BMI), basal metabolic rate, etc., based on the input physical data.

In addition, when the subject corresponds to the rapid growth period 303, a solution for increasing the growth prediction value of the rapid growth period 303 of the subject may be provided. The growth prediction value is a value corresponding to the y-axis in FIG. 2, and the solution for increasing the growth prediction value may be provided to the subject through various solution display units 90 for increasing the expected target value of the y-axis.

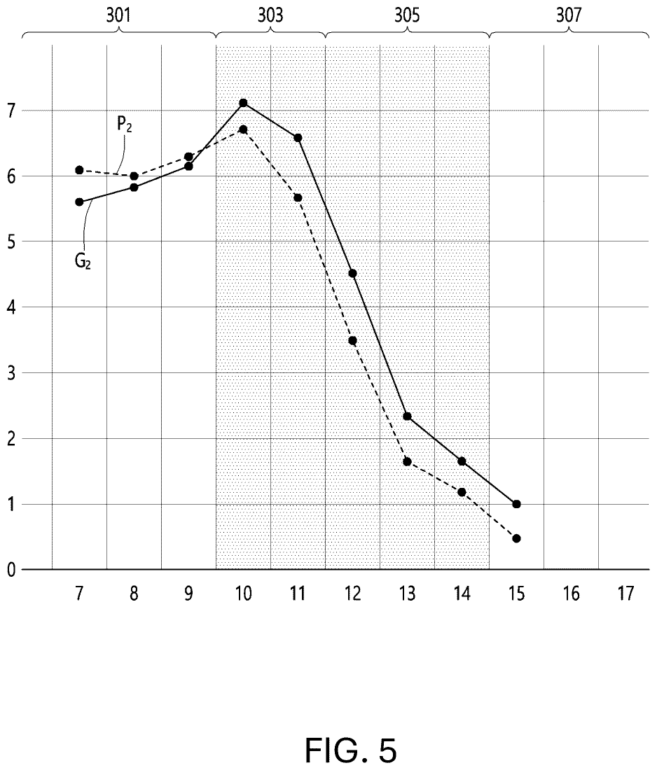

FIG. 4 is a diagram illustrating the growth in obesity and normal weight for each growth stage of a boy according to an exemplary embodiment of the present disclosure and FIG. 5 is a diagram illustrating the growth in obesity and normal weight for each stage of a girl according to an exemplary embodiment of the present disclosure.

In FIG. 4, an x-axis refers to the age, and a y-axis refers to the growth rate (cm). A solid line G1 represents the growth rate by growth stage in the case of normal weight for boys, and a dotted line P1 represents the growth rate for each growth stage in the case of obesity for boys.

In FIG. 5, an x-axis refers to the age, and a y-axis refers to the growth rate (cm). A solid line G2 represents the growth rate for each growth stage in the case of normal weight for girls, and a dotted line P2 represents the growth rate for each growth stage in the case of obesity for girls.

It will be described with reference to FIGS. 4 and 5 below.

Obesity is not a simple increase in weight, but is overweight accompanied by excessive accumulation of fat tissue in the body or a disease that is accompanied by metabolic disorders caused by the overweight. The obesity in children and adolescents medically refers to a case in which the body weight is 20% or more than a standard weight for each height in an age group from infancy to puberty.

The obesity in the infancy usually disappears after a first birthday of a child as the movement and activity of the child become more active. However, in the case of some children, obesity persists, and there are many cases where weight returns to normal but obesity recurs at school age.

75% to 80% of obesity in children and adolescents transitions to adult obesity. In addition, obesity inhibits a secretion of growth hormones.

Especially, in the case of girls, puberty is accelerated and the period of growth potential is shortened, so growth is hindered or precocious puberty is caused.

Therefore, it is necessary to provide systematic solutions to predict obesity and prevent obesity when obesity is predicted for school-age children and adolescents who are prone to obesity.

Meanwhile, obesity in children and adolescents may be divided into simple obesity for which the exact cause is not known and symptomatic obesity caused by a special causative disease, and more than 99% of childhood obesity is simple obesity.

Both boys and girls with simple obesity tend to have average height or be slightly taller than those of the same age group (a plurality of sample subjects) in the normal growth period 301, but tend to be shorter or have a lower growth rate than those of the same age group (a plurality of sample subjects) after the rapid growth period 303.

In summary, obesity in children and adolescents may be understood as a group of diseases that are accompanied by overweight or metabolic disorders resulting from a wide variety of causes. Referring to FIGS. 5 and 6, boys and girls tends to generally have average or slightly higher growth rates than the plurality of sample subjects of the same age group in the normal growth period 301, but tends to have a lower growth rate than the plurality of sample subjects of the same age group after the rapid growth period 303.

Therefore, in order to more accurately predict the obesity and generate the solution based on the growth stage, the present embodiment may classify the gender of the subject through the gender determination unit 20 based on the physical data of the subject input through the input unit 10 and then classify the growth stage in the growth stage determination unit 30 based on the classified gender, and extract the physical data, and then generate the solution in the solution generation unit 70 by considering the gender and growth stage of the subject.

More specifically, when boys and girls commonly correspond to obesity, it can be seen that the growth prediction value in the rapid growth period 303 is lower than in the normal case. Therefore, when the subject corresponds to the rapid growth period 303, the solution for increasing the growth prediction value may be provided, and may include information on adjustment of indicators that may alleviate, particularly, abnormal increases in sex hormones, including the physical data that are taken into account when the subject corresponds to the normal growth period 301.

In addition, when the subject corresponds to the decelerated growth period 305, a solution for controlling the period of the low growth period 305 of the subject may be provided. The growth stage period adjustment may be divided into cases where the physical data of the subject is located at the beginning of the decelerated growth period 305 and cases where the physical data of the subject is located in the mid-to-late part of the decelerated growth period 305 among the growth stages classified based on the input physical data of the subject.

The standard for distinguishing between the beginning and the mid-to-late part of the above-mentioned decelerated growth period 305 may be divided based on a predetermined range corresponding to the decelerated growth period 305 from the rapid growth period 303 with respect to the x-axis in FIGS. 4 to 6. Alternatively, based on whether the secondary sexual characteristics are completed based on the input physical data of the subject, when the secondary sexual characteristics are not completed, it may be classified as the beginning of decelerated growth period 305, and when the secondary sexual characteristics are completed, it may be divided as the mid-to-late part of the decelerated growth period 305.

Preferably, it is possible to determine whether the secondary sexual characteristics have been completed based on the input physical data of the subject to determine whether the current physical data of the subject is located at the beginning or mid-to-late part of the decelerated growth period 305, and when it is not possible to determine whether the secondary sexual characteristics have been completed based on the input physical data of the subject, it is possible to determine whether the physical data of the subject is located at the beginning or mid-to-late part of the decelerated growth period 305 based on the predetermined range corresponding to the decelerated growth period 305 from the rapid growth period 303.

Meanwhile, when the input physical data of the subject is located at the beginning of the decelerated growth period 305, a period adjustment solution for delaying the entry into the decelerated growth period 305 may be provided.

As described above, the secondary sex characteristics are being completed when transitioning from the rapid growth period 303 to the decelerated growth period 305, so it is possible to provide a solution for delaying the time when the secondary growth is completed, and in FIGS. 4 to 6, various solutions for moving the range of the x-axis corresponding to the decelerated growth period 305 to the right may be provided to the subject through the display unit 90. In this case, the range of the decelerated growth period 305 may increase depending on the physical data of the subject, or may decrease as the rapid growth period 303 increases.

Meanwhile, when the input physical data of the subject is located at the mid to late part of the decelerated growth period 305, the period adjustment solution for increasing the decelerated growth period 305 may be provided.

As described above, the decelerated growth period 305 refers to the time when a growth plate of the subject closes. Generally, about 50% of the growth plate closes 6 months after entering the decelerated growth period 305, and when the growth plate closes and the natural growth stops, the non-growth period 307 is entered. In this case, a solution for increasing the period of decelerated growth period 305 may be provided. In other words, various solutions for widening the range of the x-axis corresponding to the decelerated growth period 305 in FIGS. 4 to 6 may be provided to the subject through the display unit 90.

When the above-described subject corresponds to the normal growth period 301, it may include, especially, contents on adjustment of indicators that may alleviate the degree that the growth plate closes, including the physical data to be considered.

In addition, when the subject corresponds to the non-growth period 307, a solution for improving physical functions through lifestyle habits, customized exercise, posture correction, nutrient intake, etc., based on the input physical data of the subject may be provided.

In the non-growth period 307, the growth plate closes and the natural growth stops, so the solutions for improving the physical functions through the lifestyle habits, the customized exercise, the posture correction, etc., based on body weight, body fat, body water, soft lean mass, skeletal muscle mass, body mass index (BMI), basal metabolic rate, neck circumference, chest circumference, abdominal circumference, thigh circumference, arm circumference, and hip circumference, etc., of the subject may be provided or the solution for improving the physical functions through the nutrient intake, etc., based on protein, mineral content, bone tissue (bone density), etc., may be provided.

Meanwhile, in order to more accurately predict the obesity and provide the solution in the present embodiment, the gender of the subject may be classified through the gender determination unit 20, and the growth stage classification unit 31 may set the timing of the rapid growth stage differently based on the classified gender.

As described above, this is because the entry time into the rapid growth stage may be different for boys and girls. The physical data of the subject corresponding to the rapid growth stage considering the gender output from the physical data extraction unit 33 is input to the prediction unit 50 to output the prediction value for obesity.

When the subject is predicted to be obese and the gender is male, a solution for increasing the growth prediction value of the subject in the rapid growth period 303 may be provided, which is as described above.

When the subject is predicted to be obese and the gender is female, a solution for increasing the period of the rapid growth period 303 may be provided.

In more detail, referring to FIGS. 4 and 5, in the case of women, the difference in the growth prediction value in the rapid growth period 303 is smaller than that of men, but as it transitions from the rapid growth period 303 to the decelerated growth period 305, the growth rate of height decreases significantly with age.

For example, in FIG. 4, it can be seen that, in the case of a man having normal weight, the difference in the growth prediction value in the rapid growth period 303 is clearly different from the difference in the growth prediction value in the case of the normal weight G1 and the case of the obesity P1. Therefore, in the case of the obese man, there is a need to provide the solution for increasing the growth prediction value in the rapid growth period 303.

In addition, as an example, in FIG. 5, a woman having normal weight grows 7 cm in height as she grows from 9 to 10 years old, and grows about 6.5 cm in height as she grows from 10 to 11 years old.

In contrast, it can be seen that an obese woman grow approximately 6.7 cm in height from 9 to 10 years old, and approximately 5.7 cm in height from 10 to 11 years old. In FIG. 5, it can be seen that the difference in growth prediction value is relatively small when compared to the case of man.

Therefore, in the case of the obese women, it is necessary to provide the solution for increasing the period of the rapid growth period 303 to reduce the decrease range of the growth rate that occurs when transitioning from the rapid growth stage to the decelerated growth stage.

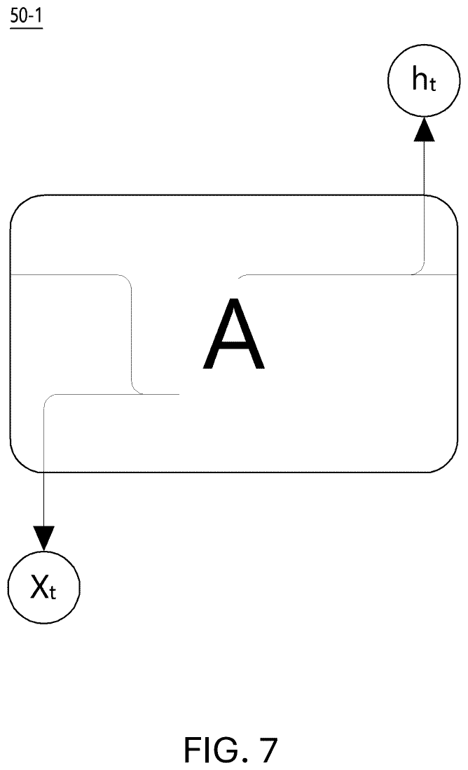

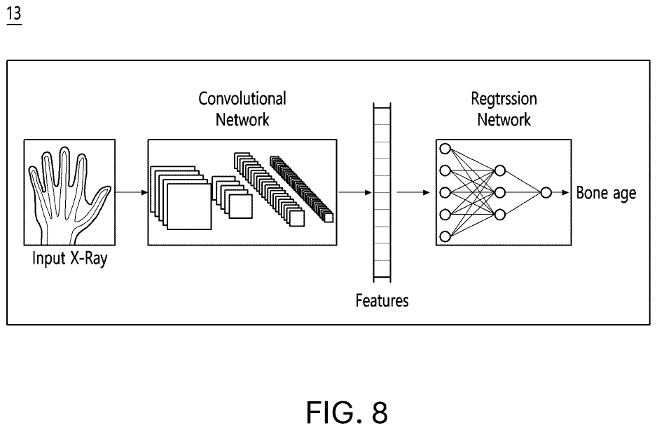

FIG. 6 is a diagram illustrating a configuration of a neural network for performing growth prediction and solution provision according to an exemplary embodiment of the present disclosure, FIG. 7 is a diagram illustrating a first neural network model according to an exemplary embodiment of the present disclosure, and FIG. 8 is a diagram illustrating a second neural network model according to an exemplary embodiment of the present disclosure.

This will be described with reference to FIGS. 6 to 8 below.

An exemplary embodiment of the present disclosure may include a first model (50-1 to 50-4) and a second model 13, and a pipeline may be built in which at least some of the output of the second model 13 is input to the first model 50.

More specifically, the first model (50-1 to 50-4) is a model that trains physical data corresponding to at least one of the plurality of growth stages as training data based on the time-series physical data for the plurality of sample subjects.

In the growth stage, the rapid growth period 303, which is the time when growth slowdown due to obesity begins, may be adopted, but as described above, any one or more of the plurality of growth stages may be adopted to more accurately predict obesity.

The first model includes LSTM neural networks 50-1, 50-2, 50-3, and 50-4 for training time-series data, and trains the LSTM neural networks 50-1, 50-2, 50-3, and 50-4 using past physical information of the plurality of sample subjects. The physical data of the current subject may be input to the trained LSTM neural networks 50-1, 50-2, 50-3, and 50-4 to output the predicted growth rate and the solution considering the growth rate.

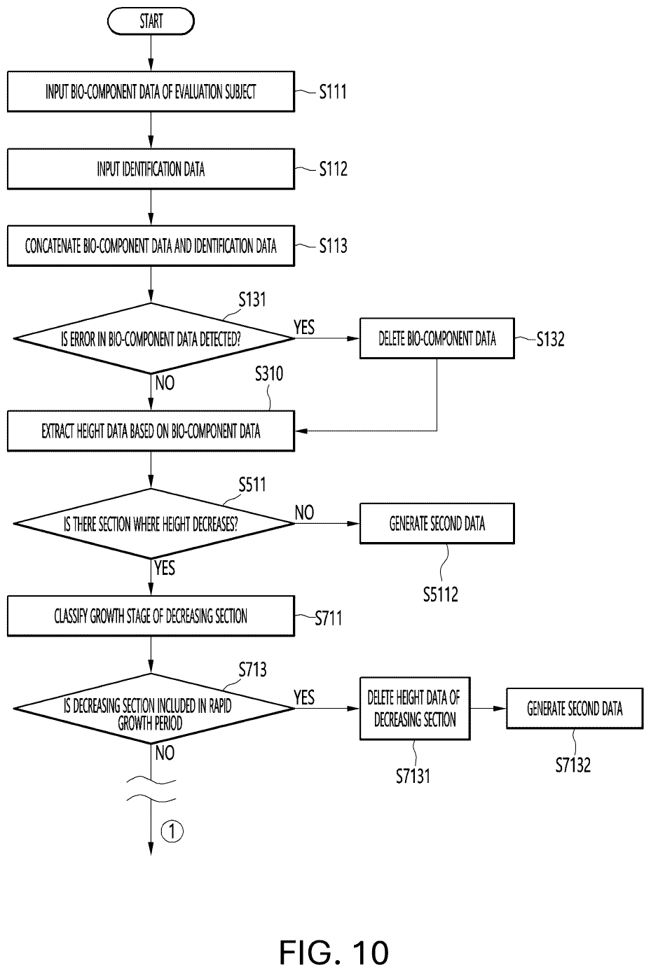

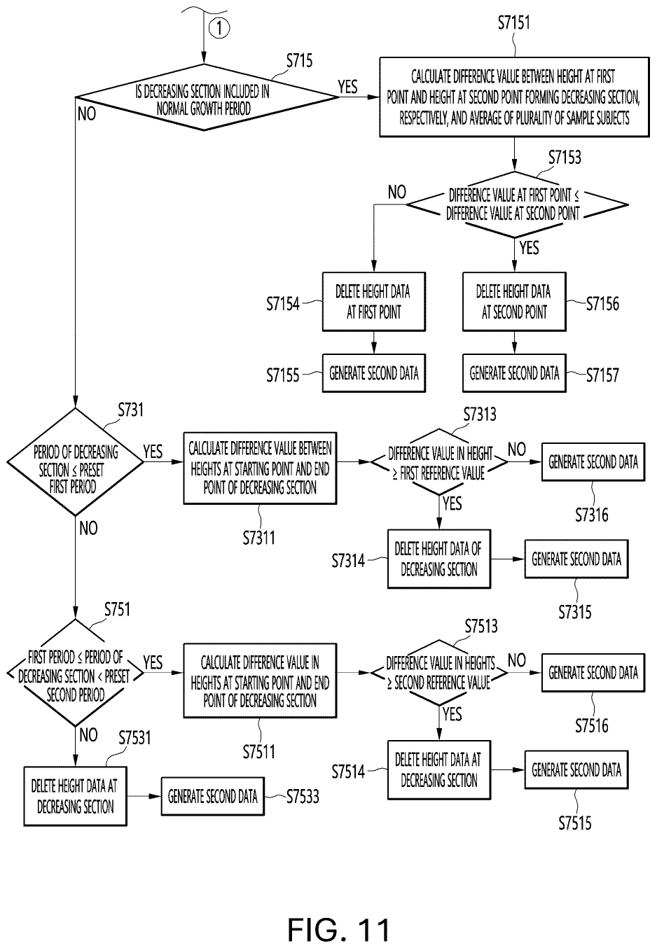

The physical data of the subject may mean the refined data that has gone through an error determination process through the data preprocessing unit 100, which will be described in more detail later with reference to FIGS. 10 and 11.

The LSTM neural networks 50-1, 50-2, 50-3, and 50-4 are trained using at least one of the physical data of the plurality of sample subjects as a default. For example, for height, training is performed with annual height data during an arbitrary period or specific growth stage, and the prediction for the next year is made and compared with actual data. By this comparison, the training set is trained as it moves into the future at random periods or in units of specific growth stages.

In addition, the LSTM neural networks 50-1, 50-2, 50-3, and 50-4 may be trained for each growth stage. Therefore, the normal growth period 301, the rapid growth period 303, the decelerated growth period 305, and the non-growth period 307 may each be trained with the past physical data of the corresponding growth stage.

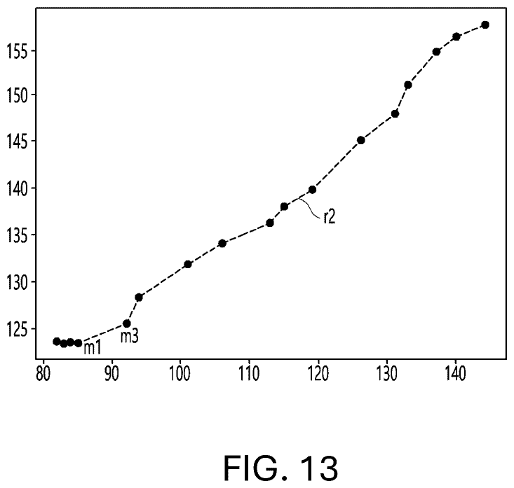

The physical data of the subject for the training may mean the refined data that has gone through an error determination process through the data preprocessing unit 100, which will be described in more detail later with reference to FIGS. 9 and 13.

Meanwhile, illustratively, in this embodiment, the time series physical information on the plurality of sample subjects is sequentially input as the training data according to age or arbitrary period, and the calculation result of the prediction value at the past point in time or growth rate may be transmitted to the growth rate prediction at the next age or arbitrary period.

Therefore, the LSTM neural networks 50-1, 50-2, 50-3, and 50-4 may not only predict the growth rate based on the current physical data, but also train the extent to which prediction results 50-1, 50-2, 50-3, and 50-4 for by various indicators at the past point in time affects the past the current growth rate prediction, so items that have a significant impact on the change in the growth rate depending on age or arbitrary period among the indicators may be extracted and reflected in the growth rate prediction.

In addition, for time series learning, it is necessary to secure the physical data of the plurality of sample subjects at regular intervals. However, as described above, it may be difficult to regularly obtain the physical data of the plurality of sample subjects depending on age or arbitrary period, so it can be used by removing outlier physical data or non-continuous physical data for each unit period and normalizing it in time.

Meanwhile, the second model 13 may derive bone maturity (age) from a carpal image using a convolution neural network trained with bone maturity data of a subject as training data.

More specifically, the convolutional neural network may include a plurality of convolution layers that creates a feature map for features in an image to be analyzed among the carpal images and a pooling layer where sub-sampling is performed between the plurality of convolutional layers to extract features at different levels for an area to be analyzed, may be inferred probabilistically through an activation function, or may derive the bone maturity through weight learning between nodes through regression analysis.

The bone maturity extracted through the second model 13 may be input to the LSTM neural networks 50-1, 50-2, 50-3, and 50-4 along with at least some of the physical data of the subject to increase the accuracy of predicting the growth rate of the subject, thereby further increasing the accuracy of predicting the obesity, precocious puberty, and growth of the subject.

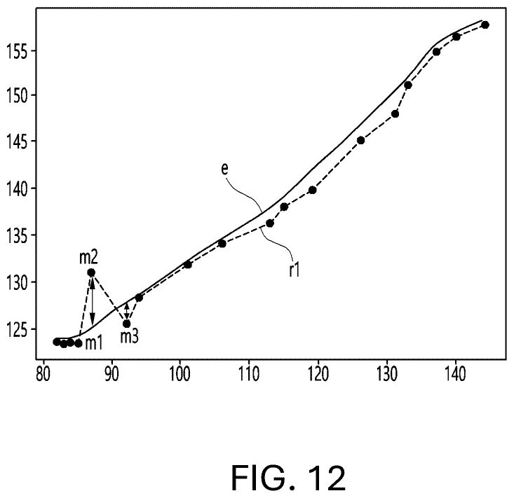

FIGS. 9 to 11 are diagrams illustrating a method of preprocessing height measurement data for growth prediction according to an exemplary embodiment of the present disclosure, and FIGS. 12 and 13 are diagrams visually illustrating a process of preprocessing input data according to an exemplary embodiment of the present disclosure.

As described above, when predicting the growth of the subject (children and adolescents) or providing the solution through the growth prediction of the subject (children and adolescents), the input physical data may have the error in the measurement process or the error in the data transmission.

Therefore, in order to more accurately predict the growth prediction or providing the solution through the growth prediction of the subject, it is preferable to input the refined data that has gone through the process of detecting and determining the above-described error data to the input unit 10.

In addition, the physical data of the plurality of sample subjects for training the prediction model of the above-described prediction unit 50 may also use the refined data that has gone through the process of detecting and determining the above-described error data.

Hereinafter, the process of detecting and determining errors in height measurement data will be described with reference to FIGS. 2, 9 to 13.

An exemplary embodiment of the present disclosure may receive the physical data (S10) to detect or determine the error in the physical data of the subject, particularly the error in the height data, and then extract the height data from the input physical data (S30).

After it is determined whether there is the section where the height decreases based on the extracted height data (S50), when there is the section where the height decreases (S50: Yes), the growth stage of the section where the height data decreases may be classified (S70). Therefore, through the step (S70), it is possible to classify whether the section where the height decreases corresponds to one of the above-described normal growth period 301, rapid growth period 303, decelerated growth period 305, and non-growth period 307.

The error of the physical data may be determined in consideration of the growth stage (S90).

More specifically, the physical data of the subject may include the identification data 1201 and the bio-component data 1101 as illustrated in FIG. 15, and the preprocessing unit 100 may receive the bio-component data 1101 of the subject (S111) through the bio-component data input unit 110, and receive the identification data 1201 of the subject through the identification data input unit 120 (S112).

The input bio-component data 1101 and the identification data 1201 may be generated as the first data 140d that is concatenated through the connection unit 140. The data 140d may also be named as first data to be distinguished from the second data refined through the data preprocessing unit 100 of the present embodiment.

When it is determined that the error is detected in the bio-component data 1101 input through the process (S131) of detecting the error in the bio-component data through the error detection unit 130 (S131: Yes), at least some of the bio-component data constituting the concatenated data 140d may be deleted (S132).

For example, when at least one of the plurality of items 1101-1 to 1101-9 that constitute the input bio-component data 1101 includes a ‘Nan’ value, the data preprocessing unit 100 of the present embodiment may delete the bio-component data 1101 through the data deletion unit 180. In this case, all items (rows including the measurement date) measured at the measurement date including the ‘Nan’ value based on the measurement date (1201-4) of the identification data may be deleted.

Alternatively, when the monthly age is 30 months or less based on the monthly age 1201-3 of the identification data, all items (rows including the measurement date) measured at the measurement date that include the ‘Nan’ value may be deleted. In more detail, when the monthly age is 30 months or less, it is a period when rapid growth of infants and toddlers occurs, and may include many errors during the measurement, so the reliability of the bio-component data is relatively lower than that of bio-component data with a high monthly age.

Therefore, when the bio-component data includes the ‘Nan’ value as described above when the monthly age is 30 months or less, all items measured at the measurement date that include the ‘Nan’ value may be deleted.

The deletion of the bio-component data described above is exemplary, and when there is an error (for example, the Nan value) in the items that constitute the bio-component data by various criteria, the error may be detected and deleted.

Meanwhile, when no error is detected in the bio-component data 1101 (S131: No), the height data 1101-2 may be extracted through the height data extraction unit 151.

It may be determined whether the decreasing section exists in the extracted height data through the decreasing section determination unit 153 (S511). When the section where the height data decreases does not exist (S511: No), since there is no error in the height data, the refined second data that may be input to the input unit 10 or the prediction model may be generated through the second data generation unit 190 (S5112).

Meanwhile, when the section where the height data decreases exists (S511: Yes), the growth stage including the section where the height data decreases may be classified through growth stage classification unit 155 (S711), and the error in the bio-component data may be determined.

More specifically, since the growth rate of children and adolescents in each growth stage is different, the change range of the height is different. For example, in general, the change range of the height in the rapid growth period 303 is greater than the change range of the height in the normal growth period 301.

Therefore, when determining the error in the height data, different criteria may be applied when the section where the height data decreases corresponds to the rapid growth period 303 and when the section where the height data decreases corresponds to the normal growth period 301.

More specifically, the present embodiment may classify the growth stage including the section where the height data decreases (S711) and then determine whether the decreasing section is included in the rapid growth period (S713).

When the decreasing section is included in the rapid growth period (S713: Yes), the data deletion unit 180 may delete all the height data forming the decreasing section (S7131) and generate the refined second data through the second data generation unit 190.

The data deleted through the data deletion section 180 may mean the height data 1101-2 among the bio-component data forming the section where the height data decreases.

Alternatively, in order to increase the data reliability, all the items (rows including the measurement date) measured at the measurement date including the height data forming the section where the height data decreases based on the measurement date 1201-4 of the identification data may be deleted. In other words, all the data in the rows including the height data forming the section where the height data decreases may be deleted.

Meanwhile, when the decreasing section is not included in the rapid growth period (S713: No), it may be determined whether the decreasing section is included in the normal growth period (S715).

The data error determination criteria for the case where the decreasing section is included in the normal growth period (S715: Yes) may be set differently from the data error determination criteria for the case where the decreasing section is included in the rapid growth period (S713: Yes).

As described above, since the change range of height in the rapid growth period 303 is greater than the change range of the height in the normal growth period 301, when the section in which the height continuously measured decreases occurs in the rapid growth period 303, all the height data forming the decreasing section may be deleted, and when the section in which the height continuously measured decreases occurs in the normal growth period 301, data having a higher probability of being error data among the height data forming the decreasing section may be deleted.

More specifically, when the decreasing section is included in the normal growth period (S715: Yes), the error determination unit 170 may calculate the difference value between the height at the first point forming the decreasing section and the height at the second point, respectively, and the average of the plurality of sample subjects. The first point and the second point may mean a starting point and an ending point of the decreasing section.

In the step (S7151), the calculated difference value at the first point and the calculated difference value at the second point are compared (S7153), and when the difference value at the first point is greater than the difference value at the second point (S7153: Yes), the height data at the first point may be deleted (S7154) and then the second data may be generated (S7155), and when the difference value at the first point is less than or equal to the difference value at the second point (S7153: No), the height data at the second point may be deleted (S7156) and then the second data may be generated (S7157).

The reason why the difference value at the second point is deleted when the difference value at the first point and the difference value at the second point are the same in the step (S7153) is that the height data at the second point is measured later in time than the height data at the first point, and therefore more approaches the rapid growth period 303, so the height data measured at the second point is highly likely to be an error.

FIGS. 12 and 13 are diagrams visually illustrating the process of preprocessing data when the section where the height data decreases is included in the general growth stage. In FIGS. 12 and 13, an X-axis means the monthly age 1201-3, a y-axis means the height 1101-2 (cm), dotted lines r1 and r2 mean the height data of the subject, and a solid line e in FIG. 12 means data that is obtained by fitting the physical data (growth curves) of the plurality of sample subjects to a specific function.

In FIG. 12, m2 indicates a starting point (first point) of the section where the height data decreases, and m3 indicates an ending point (second point) of the section where the height data decreases. The data preprocessing unit 100 of the present embodiment may calculate the difference value from the average of the plurality of sample subjects at each point m2 and m3 when the decreasing sections m2 and m3 are included in the general growth stage as illustrated in FIG. 12.

The difference value may be calculated as the difference between the average of the height data of the plurality of sample subjects corresponding to the height data at the first point m2 and the height data at the second point m3 of the subject as described above, and may also be calculated as the difference between the data obtained by fitting the physical data (growth curve) of the plurality of sample subjects with a specific function as illustrated in FIG. 12.

The physical data of the plurality of sample subjects may be pre-stored in the error determination unit 170, and the calculation of the difference between the height data of the subject and the height data of the plurality of sample subjects may be performed, or the physical data of the plurality of sample subjects is fitted with the specific function and then the calculation of the difference between the height data of the subject and the height data of the plurality of sample subjects may also be performed. Of course, it is obvious that the above-described calculation may be performed through a separate calculation unit (not illustrated) that may be included in the data preprocessing unit 100.

In this example, the data preprocessing unit 100 may delete the height data of the first point m2 through the data deletion unit 180 because the difference value calculated at the first point m2 is greater than the difference value calculated at the second point m3 (S7153: No), and then generate the refined second data through the second data generation unit 190.

Meanwhile, when the decreasing section is not included in the normal growth period (S715: No), the error determination unit 170 may compare the period of the decreasing section with the preset first period (S731). The step (S731) corresponds to one step of the decelerated growth period 305 or the non-growth period 307 because the decreasing section existing in the height data 1101-2 input through the above-described step is not included in the normal growth period 301 or the rapid growth period 303.

Therefore, in the case described above, the present embodiment may delete the data of the decreasing section based on the period of the decreasing section and the degree of reduction in the height data.

More specifically, if the period of the decreasing section is less than the preset first period (S731: Yes), the error determination unit 170 may calculate (S7311) the difference value in the height between the starting point and the ending point of the decreasing section. That is, in the step (S7311), the error determination unit 170 derives the degree (hereinafter, the difference value in the height) of reduction in the height data.

In addition, a step (S7313) of comparing the difference value in the height with the preset first reference value may be performed.

When the difference value in the height is greater than or equal to the preset first reference value (S7314: Yes), it means that the decrease range of the height is greater than the first reference value within a relatively short period (the period of the decreasing section<less than the first period), so the data preprocessing unit 100 may delete the height data of the decreasing section through the data deletion unit 180 (S7314) and generate the second data through the second data generation unit 190 (S7315).

In the step (S7314), the deleted height data may mean the height data corresponding to the starting point of the decreasing section and the height data corresponding to the ending point.

Alternatively, the height data deleted in the step (S7314) may mean either the height data corresponding to the starting point of the decreasing section or the height data corresponding to the ending point of the decreasing section. In this case, as described above, it may be natural that the height data at each point may be compared with the plurality of sample subjects and then selectively deleted based on the difference value.

In addition, as described above, the data deleted in the step (S7314) may delete, for example, not only the height data corresponding to the starting point or the ending point of the decreasing section, but also all data (rows) included in the measurement date on which the height data was measured.

In addition, when the difference value in the height is less than the preset first reference value (S7313: No), the data preprocessing unit 100 may generate the second data through the second data generating unit 190 (S7316).

For example, the first period may be set to 6 months, and the first reference value may be set to 1 cm.

Meanwhile, when the period of the decreasing section is greater than or equal to the preset first period (S731: No), the error determination unit 170 may determine whether the period of the decreasing section is included within the range of the first period and the preset second period (S751).

When the period of the decreasing section is greater than or equal to the first period and less than the second period (S751: Yes), the error determination unit 170 may calculate the difference value in the height between the starting point and the ending point of the decreasing section (S7311). That is, in the step (S7511), the error determination unit 170 derives the degree (hereinafter, the difference value in the height) of reduction in the height data.

In addition, a step (S7513) of comparing the difference value in the height with the preset second reference value may be performed.

When the difference value in the height is greater than or equal to the preset second reference value (S7314: Yes), the data preprocessing unit 100 can delete the height data of the decreasing section through the data deletion unit 180 (S7514) and generate the second data through the second data generation unit 190 (S7515).

In addition, when the difference value in the height is less than the preset second reference value (S7513: No), the data preprocessing unit 100 may generate the second data through the second data generating unit 190 (S7516).

For example, the second period may be set to 24 months, and the second reference value may be set to 0.5 cm.

The case where the period of the section where the height data decreases is included in the range set as the first period and the second period in the step (S751) means that the case where the difference in the number of measured months of the input data is greater than the case where the decreasing section is less than the first period, so as described above, it is preferable that the second reference value be set to a smaller value than the first reference value.

Meanwhile, when the monthly age of the subject is outside the range set as the first period and the second period (S751: No), since the difference in the number of measured months is relatively greater, the error determination unit 170 may determine all of the height data forming the decreasing section as errors, delete all of the height data through the data deletion unit 180 (S7531), and then generate second data through the second data generation unit 190 (S7533).

Hereinabove, the present disclosure has been described with reference to exemplary embodiments. All exemplary embodiments and conditional illustrations disclosed in the present disclosure have been described to intend to assist in the understanding of the principle and the concept of the present disclosure by those skilled in the art to which the present disclosure pertains.

Therefore, it will be understood by those skilled in the art to which the present disclosure pertains that the present disclosure may be implemented in modified forms without departing from the spirit and scope of the present disclosure.

Therefore, the embodiments disclosed herein should be considered in an illustrative aspect rather than a restrictive aspect. The scope of the present disclosure should be defined by the claims rather than the above description, and equivalents to the claims should be interpreted to fall within the present disclosure.

Meanwhile, the methods according to various exemplary embodiments of the present disclosure described above may be implemented as programs and be provided to servers or devices. Therefore, the respective apparatuses may access the servers or the devices in which the programs are stored to download the programs.

In addition, the methods according to various exemplary embodiments of the present disclosure described above may be implemented as programs and be provided in a state in which it is stored in various non-transitory computer-readable media. The non-transitory computer readable medium is not a medium that stores data for a while, such as a register, a cache, a memory, or the like, but means a medium that semi-permanently stores data and is readable by an apparatus. In detail, the various applications or programs described above may be stored and provided in the non-transitory computer readable medium such as a compact disk (CD), a digital versatile disk (DVD), a hard disk, a Blu-ray disk, a universal serial bus (USB), a memory card, a read only memory (ROM), or the like.

Although the embodiments of the disclosure have been illustrated and described hereinabove, the disclosure is not limited to the specific embodiments described above, and may be variously modified by those skilled in the art to which the disclosure pertains without departing from the scope and spirit of the disclosure as claimed in the claims. These modifications should also be understood to fall within the technical spirit and scope of the disclosure.

1. A method of preprocessing height measurement data for growth prediction performed by computing device, comprising:
receiving time-series bio-component data of a subject;
receiving identification data of the subject;
extracting height data from the bio-component data;
determining whether there is a decreasing section within a section where the height data is input; and
when there is the section where the height data decreases, determining an error in the height data based on a growth stage in which the decreasing section is included.

2. The method of claim 1, further comprising generating first data by concatenating the time-series bio-component data of the subject and the identification data of the subject.

3. The method of claim 2, further comprising, after generating the first data, deleting the bio-component data when a preset value is included in the bio-component data.

4. The method of claim 1, wherein the growth stage includes a normal growth period, a rapid growth period, a decelerated growth period, and a non-growth period, and is classified based on a monthly age of the identification data.

5. The method of claim 4, wherein, in the determining of the error, when the decreasing section is included in the rapid growth period, the height data of the decreasing section is deleted.

6. The method of claim 4, wherein the determining of the error includes calculating a difference between height data at any first point and height data at a second point, respectively, forming the decreasing section and an average data value of the corresponding monthly age of a plurality of sample subjects, when the decreasing section is included in the normal growth period.

7. The method of claim 6, wherein the determining of the error further includes comparing the difference value calculated at the first point with the difference value calculated at the second point.

8. The method of claim 7, wherein, in the determining of the error, when the difference value calculated at the first point is smaller than the difference value calculated at the second point, the height data at the second point is deleted.

9. The method of claim 7, wherein, in the determining of the error, when the difference value calculated at the first point is greater than or equal to the difference value calculated at the second point, the height data at the first point is deleted.

10. The method of claim 4, wherein, in the determining of the error, when the decreasing section is included in the decelerated growth period or the non-growth period, the data of the decreasing section is deleted based on a period of the decreasing section and a degree of decrease in height.

11. A program stored in a computer-readable recording medium including a program code for executing the method of preprocessing height measurement data for growth prediction according to claim 1.

12. A computer-readable recording medium on which a program for executing the method of preprocessing height measurement data for growth prediction according to claim 1 is recorded.

13. An apparatus for preprocessing height measurement data for growth prediction, comprising:
a first input unit that receives time-series bio-component data of a subject;
a second input unit that receives identification data of the subject;
a height data extraction unit that extracts height data from the bio-component data;
a decreasing section determination unit that determines whether there is a decreasing section within a section where the height data is input;
a growth stage classification unit that classifies a growth stage including the decreasing section when there is the section where the height data decreases; and
an error determination unit that determines an error in the height data based on the growth stage including the decreasing section.

14. The apparatus of claim 13, further comprising a connection unit that connects data input through the first input unit and the second input unit to generate first data.

15. The apparatus of claim 14, further comprising an error detection unit that deletes the bio-component data when a preset value is measured in the bio-component data among the first data.
